(19) United States
(12) Patent Application Publication
(10) Pub. No.: US 2020/0138642 A1
(43) Pub. Date: May 7, 2020

(54) Absorbent Articles

(71) Applicant: ALYK, Inc., New York, NY (US)

(72) Inventors: Sarah Wagner, New York, NY (US); Monica Belsito, New York, NY (US); Nick Pendleton, Fairfield, OH (US); Al Fischer, Fairfield, OH (US)

(73) Assignee: ALYK, Inc., New York, NY (US)

(21) Appl. No.: 16/667,028

(22) Filed: Oct. 29, 2019

Related U.S. Application Data
(60) Provisional application No. 62/755,058, filed on Nov. 2, 2018.

(51) Int. Cl. A61F 13/511; A61F 13/472; A61F 13/476; A61F 13/15

(52) U.S. Cl. CPC A61F 13/51121; A61F 13/47272; A61F 2013/51042; A61F 13/15203; A61F 13/476

(57) ABSTRACT

An absorbent article such as a sanitary napkin, incontinence pad and breast pad.

This application claims the benefits of U.S. Provisional Patent Application No. 62/755,058 filed on Nov. 2, 2018, the contents of which is incorporated herein by reference.

### FIELD OF THE INVENTION

The present invention relates to absorbent articles such as sanitary napkins, incontinence pads, breast pads, meat, poultry and fish storage and packaging pads, pet training pads, mop pads as well as sanitary napkins with a unique shape and absorbent material.

### BACKGROUND OF THE INVENTION

Absorbent articles such as sanitary napkins, incontinence pads and breast pads are designed to absorb body liquids such as blood, urine and lacteal fluid. The design, shape and structure of the absorbent article will vary depending upon the specific use, however, absorbent articles typically have three (3) basic design elements: (i) a top sheet or surface that is in contact with the user's skin or body; (ii) a bottom/backing sheet or surface that is opposite the top sheet and contacts the user's clothing or garment; and (iii) an absorbent core between the top sheet and backing sheet. The top sheet should direct or wick the body liquids away from the user's body and into the absorbent core. The backing sheet should prevent the migration of the body liquids from the absorbent core to the user's clothing or garment. The absorbent core should absorb or hold the body liquids.

Numerous variations of the absorbent article basic structure have been proposed over the years including modifications to the composition of the various layers, modifications to the shape of the various layers, addition of further layers such as acquisition distribution layers, fluid management layers and/or wicking layers and the formation of structural elements such as openings, channels, depressions and/or protrusions in one or more of the various layers of the absorbent article that aid in directing body liquids to desired sections of the absorbent article.

Examples of various absorbent articles and some of the aforementioned modifications to the basic structure can be found in U.S. Pat. Nos. 3,442,268; 4,074,721; 4,687,478; 4,690,680, 5,149,334; 5,704,932; 6,965,058; 8,183,430; 9,259,361 and U.S. Patent Application Publication Nos. 2003/0220048 and 2016/0228304.

Although the prior art absorbent articles work for their intended function, there is always a need to improve upon the prior designs. In particular there is a need for a more comfortable and effective design which will allow for better absorbency, preferably using natural or non-synthetic materials such as plant based materials.

### SUMMARY OF THE INVENTION

The foregoing needs and others are met by a first aspect of the present invention, which is an absorbent article comprising a top sheet, a backing sheet and an absorbent core sandwiched between the top sheet and backing sheet wherein the top sheet is a mixture of hydrophobic and hydrophilic cotton. In certain embodiments of this first aspect of the present invention, the backing sheet is a water and body liquid impermeable barrier, preferably a polymeric layer or sheet comprising polyethylene, polylactic acid or any other conventionally known water impermeable backing materials. In certain embodiments of this first aspect of the invention, the absorbent article may further comprise one or more acquisition distribution layers, one or more fluid management layers and/or one or more wicking layers or sheets, preferably between the top sheet and the absorbent core. The acquisition distribution layers, fluid management layers and/or wicking layers will prevent or inhibit the liquids in the absorbent core from rewetting or returning to the top sheet. In certain embodiments of this first aspect of the invention, the absorbent article may further comprise an absorbent core comprising biodegradable materials such as cellulosic or plant based absorbent materials. In certain embodiments of this first aspect of the present invention, the absorbent article and preferably the absorbent core of the absorbent article may comprise a superabsorbent polymer. Alternatively the absorbent article or the absorbent core of the absorbent article is free or substantially free of superabsorbent polymeric material. In further embodiments of this first aspect of the present invention, the top sheet is in direct contact with the absorbent core and the top sheet and absorbent core comprise biodegradable materials such as cellulosic or plant based materials and are preferably free of synthetic polymeric materials. In still further embodiments of this first aspect of the present invention, the absorbent article is free of any acquisition distribution layers, fluid management layers and/or wicking layers or sheets and particularly free of any synthetic acquisition distribution layers, synthetic fluid management layers and/or synthetic wicking layers.

Unless specifically stated, the absorbent article in accordance with the first aspect of the invention may be any type of absorbent article such as a pad used to store meat, poultry or fish for transport and sale, pet training pads that are used to house train pets such as dogs and puppies, mop pads, sanitary napkins, incontinence pads and breast pads.

The foregoing needs and others are also met by a second aspect of the present invention, which is a shaped sanitary napkin that is comfortable, exhibits improved absorbency preferably using natural or non-synthetic materials such as plant based materials. More specifically, the second aspect of the present invention is a sanitary napkin comprising a top sheet, an absorbent core and backing sheet. The sanitary napkin and particularly the absorbent core may exhibit a modified hourglass shape comprising a front bulb area, a rear bulb area and a neck area connecting the front bulb area and the rear bulb area wherein the maximum width of the front bulb area is less than or equal to the maximum width of the rear bulb area and the minimum width which is present in the neck area is less than the maximum widths of either the front bulb area or rear bulb area. The longitudinal distance from the terminal end of the front bulb area to the minimum width of the neck area or mid-point of the neck area is less than the longitudinal distance from the terminal end of the rear bulb area to the minimum width of the neck area or mid-point of the neck area in certain embodiments of the modified hourglass shape. The absorbent core comprises a material for absorbing and retaining menstrual fluid or other body liquids. The sanitary napkin may also comprise two side panels or wings that extended laterally from the neck region of the modified hourglass shape.

When the sanitary napkin of the second aspect of the present invention is used, it is applied to the inner surface of the user's undergarment, i.e. the undergarment surface that will contact the skin of the user, so the front bulb area is oriented in the front portion of the undergarment, the neck area is in the crotch portion of the undergarment and the rear bulb area is oriented in the rear portion of the undergarment. The top sheet will be in contact with the user and the backing sheet will be in contact with the inner surface of the undergarment. If present, the side panels or wings will extend through the leg openings of the undergarment and be folded so that they contact the outer surface of the crotch portion of the undergarment.

The foregoing needs and others are further met by a third aspect of the present invention which is a shaped sanitary napkin with unique side panel or wing dimensions that allow the side panels or wings to extend through the leg openings of the user's undergarment and be folded so that they contact the outer surface of the crotch portion of the undergarment without adversely affecting the comfort of the user or effectiveness of the sanitary napkin.

The foregoing needs and others are still further met by a fourth aspect of the present invention, which is a shaped sanitary napkin as described in the second aspect of the present invention and that employs: (i) the structure and composition features described above and below in the description of the first aspect of the present invention; (ii) the unique side panel or wing dimensions described above and below in the description of the third aspect of the present invention or (iii) a combination of the structure and composition features described above and below in the descriptions of the first aspect of the present invention and the unique side panel or wing dimensions described above and below in the third aspect of the present invention.

In all embodiments and aspects of the present invention, the absorbent core, which is located between the top sheet and the backing sheet, should comprise at least 50%, or more of natural or non-synthetic materials, preferably plant based materials and most preferably cotton. The absorbent core may be a single mass of absorbent material or comprise one or more layers of absorbent material.

### BRIEF DESCRIPTION OF THE DRAWINGS

FIG. 1 shows a top view of one embodiment of a sanitary napkin of the present invention without a top sheet.

FIG. 2 shows a top view of another embodiment of a sanitary napkin of the present invention without a top sheet.

### DETAILED DESCRIPTION OF THE INVENTION

Unless defined otherwise, all technical and scientific terms used herein have the same meaning as commonly understood by one of ordinary skill in the art to which the present disclosure belongs.

As used herein, the term “about” when used before a numerical designation indicates approximations which may vary by (+) or (−) 10%, 5% or 1% unless specifically stated.

As used herein the term “substantially free” means 15 wt % or less of the identified material, component or ingredient, preferably 10 wt % or less, 9 wt % or less, 8 wt % or less, 7 wt % or less, 6 wt % or less, 5 wt % or less, 4 wt % or less, 3 wt % or less, 2 wt % or less, or 1 wt % or less of the identified material. For example an absorbent core that is substantially free of a superabsorbent polymer is understood to mean the material between the top sheet and backing sheet contains 15 wt % or less of a superabsorbent polymeric material based on the total dry weight of the absorbent core, preferably 10 wt % or less, 9 wt % or less, 8 wt % or less, 7 wt % or less, 6 wt % or less, 5 wt % or less, 4 wt % or less, 3 wt % or less, 2 wt % or less, or 1 wt % or less of a superabsorbent polymeric material based on the total dry weight of the absorbent core. The term “free” means 0 wt % of the identified material, component or ingredient.

It must be noted that as used herein and in the appended claims, the singular forms “a,” “an,” and “the” include plural referents unless the context clearly dictates otherwise.

The present invention is an absorbent article such as a pad used to store meat, poultry or fish for transport and sale, pet training pads that are used to house train pets such as dogs and puppies, mop pads, sanitary napkins, incontinence pads and breast pads comprising a top sheet, a backing sheet and an absorbent core sandwiched between the top sheet and backing sheet.

The top sheet may comprise any material commonly used in the absorbent article field. The top sheet should be compliant, soft and nonirritating to the user's skin. Examples of suitable materials for the top sheet include, but are not limited to, woven and nonwoven polyester, polypropylene, nylon, rayon or the like, particularly in the form of formed or apertured thermoplastic films, such as those described in U.S. Pat. Nos. 4,324,246 and 4,342,314 which are incorporated herein by reference. Other known materials that can be employed in the top sheet include those made from woven and nonwoven cellulosic materials. The top sheet should be permeable or pervious to liquids such as water and body liquids. Additional examples and descriptions of top sheets can be found in U.S. Pat. Nos. 6,486,379, 6,494,871 and 7,994,386 which are incorporated herein by reference.

In certain aspects and embodiments of the present invention the top sheet is free or substantially free of synthetic polymeric materials, and is preferably formed from woven or nonwoven natural materials such a cellulosic or plant based materials. In a preferred embodiment the top sheet is a mixture of hydrophobic and hydrophilic cotton wherein the sheet comprises about 20 wt % to about 95 wt % of hydrophobic cotton based on the total dry weight of the top sheet, preferably at least 30 wt % to about 90 wt % of hydrophobic cotton based on the total dry weight of the top sheet, more preferably at least 35 wt % to about 85 wt % of hydrophobic cotton based on the total dry weight of the top sheet, and most preferably about 40 wt % to about 80 wt % of hydrophobic cotton based on the total dry weight of the top sheet. In certain embodiments the top sheet should comprise at least 30 wt %, 35 wt %, 40 wt %, 45 wt %, 50 wt %, 55 wt %, 60 wt %, 65 wt %, 70 wt %, 75 wt % 80 wt %, 85 wt %, or 90 wt % based on the total dry weight of the top sheet of hydrophobic cotton. In this preferred embodiment the top sheet should comprise about 80 wt % to about 5 wt % of hydrophilic cotton based on the total dry weight of the top sheet, preferably at least 70 wt % to about 10 wt % of hydrophilic cotton based on the total dry weight of the top sheet, more preferably at least 65 wt % to about 15 wt % of hydrophilic cotton based on the total dry weight of the top sheet, and most preferably about 60 wt % to about 20 wt % of hydrophilic cotton based on the total dry weight of the top sheet. In certain embodiments the top sheet should comprise at least 10 wt %, 15 wt %, 20 wt %, 25 wt %, 30 wt %, 35 wt %, 40 wt %, 45 wt %, 50 wt % 55 wt %, 60 wt %, 65 wt % or 70 wt % based on the total dry weight of the top sheet of hydrophilic cotton.

Hydrophobic cotton maybe be cotton that it is treated or modified with a hydrophobic imparting substance such as a fluorocarbon, siloxane, fatty acid, fatty acid ester, waxes or mixtures thereof. Treated or modified hydrophobic cottons are well known in the art and commercially available. Some examples of treated or modified hydrophobic cottons can be found in U.S. Pat. Nos. 3,535,156; 4,296,224; 4,467,013; 6,541,138; 7,531,219 and Chinese Patent Application Publication No. CN 105220475 which are incorporated herein by reference. Hydrophobic cotton may also be cotton that is natural or that has not been treated with additional chemicals. Examples of natural or untreated hydrophobic cotton include but are not limited to TRUE COTTON™, commercially available from TJ Beal Cotton Manufacturer & Supplier of Greenwood, Miss. USA. The TRUE COTTON™ material is prepared by a dry process which utilizes zero water, chemicals or process heat to prepare the fibers. Although the hydrophobic cotton has been cleaned, preferably by mechanical means, the hydrophobic cotton fibers have not been bleached or chemically altered thereby keeping the natural waxes and oils on the cotton fibers.

Examples of hydrophilic cotton include cotton that has been washed, bleached or chemically altered to remove some or all of the natural waxes and oils. Hydrophilic cotton is well known and commercially available. Examples of hydrophilic cotton and/or bleached cotton suppliers include but are not limited to GP Lumberton Cellulose LLC of Lumberton, N.C. USA, Barnhardt Mfg. Co. North Carolina, USA and WPT of Beaver Dam, Ky. USA.

In certain embodiments of the present invention, the top sheet of the absorbent article is a nonwoven sheet comprising a blend of 35% to 90% hydrophobic cotton and 10% to 65% hydrophilic cotton, preferably 40% to 85% hydrophobic cotton and 15% to 60% hydrophilic cotton and more preferably 45% to 80% hydrophobic cotton and 20% to 55% hydrophilic cotton and most preferably about 50% to about 75% hydrophobic cotton and about 25% to about 50% hydrophilic cotton wherein the forgoing percentages are based on the total amount of cotton in the top sheet. In certain embodiments the top sheet comprises only the blend of hydrophobic cotton and hydrophilic cotton. In certain embodiments the top sheet will comprise the blend of hydrophobic and hydrophilic cotton and 10 wt % or less, 9 wt % or less, 8 wt % or less, 7 wt % or less, 6 wt % or 5 wt % or less, 4 wt % or less, 3 wt % or less, 2 wt % or less, 1 wt % or less based on the total dry weight of the top sheet of a non-cotton material such as a binder.

When the top sheet is a blend of hydrophobic and hydrophilic cotton, it should have a dry basis weight of at about 20 to 80 gsm (grams per square meter), preferably a basis weight of about 22.5 to 70 gsm and more preferably a basis weight of about 25 to about 60 gsm.

The absorbent articles prepared in accordance with the present invention further comprise an absorbent core comprising a material that will absorb fluids such as water and body liquids such as blood and urine. The core material may comprise any material commonly used in the art for such purpose such as those described in U.S. Pat. Nos. 4,690,680; 5,149,334; 6,514,602; and 8,329,979; and U.S. Patent Application Publication No. 2017/0002098. Examples of suitable absorbent materials include, but are not limited to, cotton fibers or cotton lintels; creped cellulose wadding; meltblown polymers including coform; chemically stiffened, modified or cross-linked cellulosic fibers; hemp fibers, cannabis fibers, synthetic fibers such as crimped polyester fibers; peat moss; tissue including tissue wraps and tissue laminates; absorbent foams; absorbent sponges; superabsorbent polymers (in fibrous and particulate form); absorbent gelling materials; or any equivalent material or combinations of materials, or mixtures of these. The core material may comprise a single material or a combination of materials. In certain embodiments the core material comprises a combination of cotton and plant or animal based superabsorbent material, preferably a charged modified/cross linked biopolymer as described in U.S. Patent Application Publication No. 2017/0002098 which is incorporated herein by reference. In certain embodiments, the core comprises at least 50 wt %, 55 wt %, 60 wt %, 65 wt %, 70 wt %, 75 wt %, 80 wt %, 85 wt %, 90 wt %, 95 wt %, or 100 wt % of one or more natural or non-synthetic materials such as plant based materials. In other embodiments of the present invention the core comprises at least 60 wt %, 65 wt %, 70 wt %, 75 wt %, 80 wt %, 85 wt %, 90 wt %, 95 wt %, or 100 wt % cotton, preferably hydrophilic cotton.

In certain embodiments, the core comprises one or more layers of cotton, preferably at least two or more layers, three or more layers, or four or more layers wherein one or more of the layers may optionally be modified with a superabsorbent material, which may be a synthetic superabsorbent material such as a material obtained from a petrochemical or a non-synthetic or natural superabsorbent material. If a superabsorbent material is used it is preferably a plant or animal based superabsorbent material. The modification with the superabsorbent material may be accomplished by any means known in the art such as co-forming the cotton layer with the superabsorbent material, coating the superabsorbent material onto the cotton layer, spraying, shaking or sprinkling the superabsorbent material onto the cotton layer, or any combination thereof. In some embodiments the core comprises two or three layers of cotton fibers wherein at least one of the cotton layers has been modified with a superabsorbent material, preferably a plant or animal based superabsorbent material. If multiple layers of material such as multiple cotton layers are used to form the absorbent core of the absorbent article, the layers may be adhered to each other with a suitable adhesive. These adhesive layers are optional and the adhesive may be any type of adhesive commonly used in the industry. Examples are provided in U.S. Pat. Nos. 4,687,478, 4,690,680 and 8,329,979 which are incorporated herein by reference. The individual cotton layers may include a binder which is non-toxic and will aid in holding the individual cotton fibers in the layer or mat form. If a binder is present in the cotton layer it should comprise 10% or less, 9% or less, 8% or less, 7% or less, 6% or less, 5% or less, 4% or less, 3% or less, 2% or less, 1% or less or 0% of the total dry weight of the cotton layer. In certain embodiments, the cotton employed in the core is free of a binder and may be prepared by any known process such as hydro-entangled.

In some embodiments of the present invention, the core is free or substantially free of synthetic materials including superabsorbent polymeric material. In these embodiments the core comprises biodegradable materials such as cellulosic or plant based absorbent materials, preferably cotton. Examples of cotton materials that may be used in the core include nonwoven, carded products comprising about 95 wt % or more of a hydrophilic cotton and about 5 wt % or less of a binder such as a acetate copolymer binder. Alternatively, the cotton materials that may be used in the core includes nonwoven, carded products comprising 100 wt % hydrophilic cotton such as a 100 wt % hydro-entangled hydrophilic cotton.

The absorbent core may exhibit a total dry basis weight ranging from 100 gsm to 500 gsm, preferably 150 gsm to 450 gsm and most preferably about 200 gsm to about 400 gsm. The foregoing basis weight ranges are preferably based on the basis weight at the center of the absorbent core. The total basis weight may be obtained from the sum of two or more layers of absorbent material, such as two or more layers of the cellulosic or plant based materials.

The absorbent articles of the present invention also comprise a backing sheet that is a water and body liquid impermeable barrier. The backing sheet may comprise one or more layers, preferably one or more thin plastic films such as those described in U.S. Pat. Nos. 3,881,489; 3,989,867; 4,690,680; 5,149,334; and 6,514,602 which are incorporated herein by reference. Preferably the backing sheet is a thermoplastic film such as a polyethylene film, preferably a polyethylene that is derived from plant based material rather than petrochemicals, having a thickness of from about 0.075 mils to about 1.25 mils. Alternatively, the backing sheet may be a polylactic acid or PLA film having a thickness of from about 0.075 mils to about 1.25 mils.

The present invention includes a shaped sanitary napkin comprising a top sheet, an absorbent core, and a backing sheet, wherein the sanitary napkin, and the absorbent core in particular, is a modified hourglass shape comprising a front bulb area, a rear bulb area, and a neck area connecting the front bulb area and the rear bulb area. The front bulb area exhibits a maximum width that is less than or equal to the maximum width of the rear bulb area, and a minimum width, which defines the neck area, that is less than the maximum width of either the front bulb area or rear bulb area. The longitudinal distance from the terminal end of the front bulb area to the minimum width or mid-point of the neck area is less than the longitudinal distance from the terminal end of the rear bulb area to the minimum width or mid-point of the neck area in certain embodiments of the modified hourglass shape. The sanitary napkin may also comprise two side panels or wings that extended laterally from the neck area of the hourglass shape.

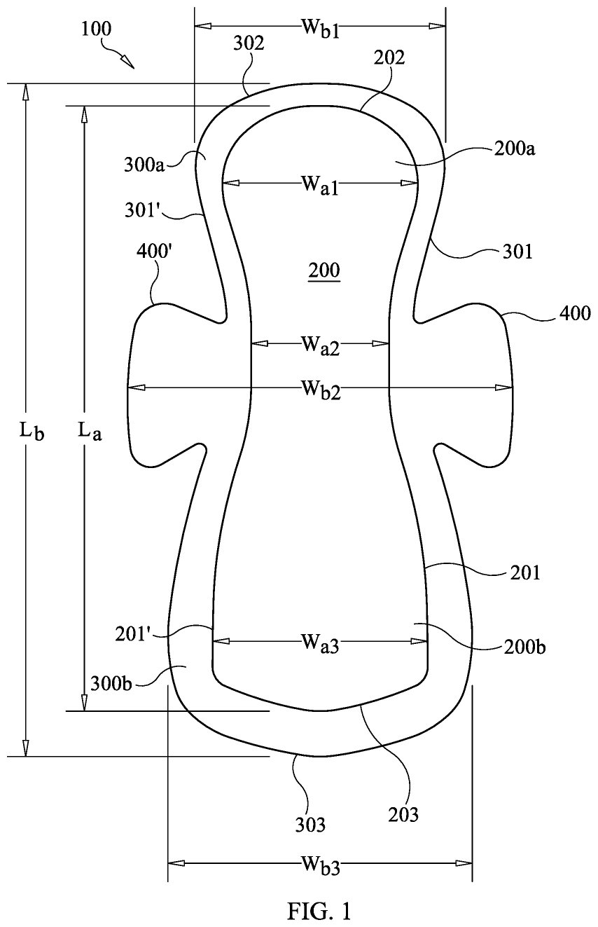

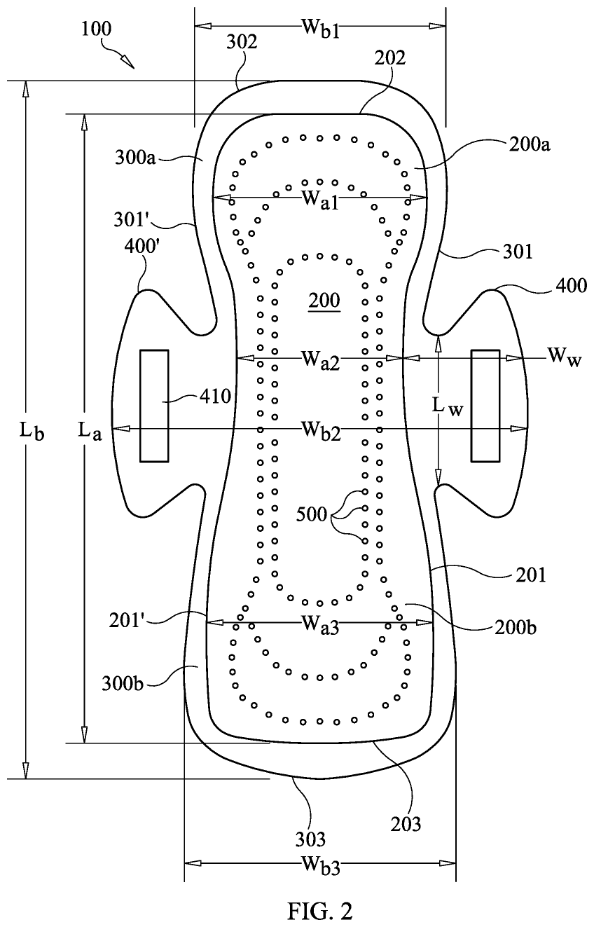

FIGS. 1 and 2 show embodiments of the shaped sanitary napkin 100 without a top sheet, comprising an absorbent core 200 in a modified hourglass shape wherein the maximum width Wₐ₁ of the front bulb area 200a is less than or equal to the maximum width Wₐ₃ of the rear bulb area 200b and the width Wₐ₂ of the neck area is less than the maximum width Wₐ₁ and Wₐ₃ of the front bulb area 200a and the rear bulb area 200b. In certain embodiments the longitudinal or outer edges 201 and 201′ of the absorbent core 200 are concave in shape, preferably curving inwardly toward a central or substantially central region of the absorbent zone 200 from points on the outer edge of the absorbent core 200 corresponding to the maximum width of the front bulb area 200a and the rear bulb area 200b to the apex of the concave curve corresponding to the minimum width of the neck area. The absorbent core 200 also comprises a front or terminal edge 202 and a rear or terminal edge 203. The front edge 202 and the rear edge 203 may be any conventional shape including but not limited to straight, angular, domed, or convex shape. The front edge 202 and rear edge 203 may be the same or different shapes. In certain embodiments, the front edge 202 and the rear edge 203 are domed or convex in shape.

In certain embodiments:
(i) the maximum width Wₐ₁ of the front bulb area 200a may range from about 50 mm to about 110 mm, preferably about 55 mm to about 100 mm, more preferably about 57 mm to about 95 mm and most preferably about 60 mm to about 90 mm;
(ii) the maximum width Wₐ₃ of the rear bulb area 200b may range from about 50 mm to about 120 mm, preferably about 55 mm to about 110 mm, more preferably about 57 mm to about 105 mm and most preferably about 60 mm to about 100 mm; and
(iii) the width Wₐ₂ of the neck area may range from about 30 mm to about 75 mm, preferably about 35 mm to about 70 mm, and most preferably about 40 mm to about 65 mm,
   with the proviso that Wₐ₃ is greater than or equal to Wₐ₁ and Wₐ₁ and Wₐ₃ are greater than Wₐ₂. In certain embodiments the ratio of Wₐ₁/Wₐ₂ can range from about 1.1 to about 2.0, preferably about 1.2 to about 1.9, and most preferably about 1.3 to about 1.8; the ratio of Wₐ₃/Wₐ₂ can range from about 1.1 to about 2.0, preferably about 1.2 to about 1.9, and most preferably about 1.3 to about 1.8; and the ratio of Wₐ₃/Wₐ₁ can range from about 1.0 to about 1.8, preferably about 1.0 to about 1.7, and most preferably about 1.0 to about 1.6.

In one embodiment Wₐ₁ is about 65-90 mm, Wₐ₂ is about 40-65 mm, and Wₐ₃ is about 65-100 mm.

The longitudinal length Lₐ of the absorbent zone 200 is about 200 mm to about 300 mm, preferably about 200 mm to about 290 mm, and most preferably about 200 mm to about 280 mm.

In certain embodiments:
(i) the longitudinal length from the apex or center of the front edge 202 to the mid-point of the neck region, crotch region, or narrowest region, i.e., minimum width Wₐ₂ of the shaped sanitary napkin, (L_{a front}) is about 80 mm to about 140 mm, preferably about 85 mm to about 137 mm, more preferably about 87 mm to about 135 and most preferably about 90 mm to about 130 mm; and
(ii) the longitudinal length from the apex or center of the rear edge 203 to the mid-point of the neck region, crotch region, or narrowest region, i.e., minimum width Wₐ₂ of the shaped sanitary napkin, (L_{a rear}) is about 80 mm to about 180 mm, preferably about 85 mm to about 175 mm, more preferably about 90 mm to about 170 mm and most preferably about 95 mm to about 165 mm, with the proviso that L_{a rear} is longer than or equal to L_{a front} and preferably L_{a rear} is longer than L_{a front}.

In certain embodiments the sanitary napkin may have an elongated neck or crotch region. As used herein an elongated neck or crotch region is a region corresponding to the crotch area of a user's undergarment and wherein Wₐ₂ is same or substantially the same dimension (i.e., ±5-10%) in the elongated neck of crotch region. In the embodiments with the elongated neck or crotch region, the L_{a front} and L_{a rear} lengths are measured from the apex or center of the front or rear bulb edges 202 and 203 to the mid-point of the elongated neck or crotch region.

In some embodiments, as shown in FIG. 2, the L_{a front} and L_{a rear} lengths are measured from the apex or center of the front or rear bulb edges 202 and 203 to the mid-point of length L_{w} which is the longitudinal length of the wings 400, 400′ that corresponds to the area where the wings attach to or extend from the sanitary napkin and the area in which the wings fold around the crotch of the user's undergarment.

In one embodiment L_{a rear} is about 100-160 mm and L_{a front} is about 90-130 mm.

As further shown in FIGS. 1 and 2 the shaped sanitary napkin 100 comprises a backing sheet 300 which may also be in a modified hourglass shape wherein the maximum width W_{b1} of the front bulb area 300a is less than or equal to the maximum width W_{b3} of the rear bulb area 300b. In certain embodiments the longitudinal or outer edges 301 and 301′ of the backing material 300 are concave in shape, preferably curving inwardly toward the center of the absorbent core 200 from points on the outer edge of the backing sheet corresponding the maximum width W_{b1} and W_{b3} of the front bulb area 300a and rear bulb area 300b to the apex of the concave curve corresponding to the minimum width of the neck region. The backing sheet 300 also comprises a front or terminal edge 302 and a rear or terminal edge 303. The front edge 302 and the rear edge 303 may be any conventional shape including but not limited to straight, angular, domed, or convex shape. The front edge 302 and rear edge 303 may be the same or different shapes. In certain embodiments, the front edge 302 and the rear edge 303 are domed or convex in shape. The backing sheet 300 may be slightly larger than the absorbent core 200 but comprise substantially the same shape as the absorbent core 200.

In certain embodiments:
(i) the maximum width W_{b1} of the front bulb area 300a may range from about 60 mm to about 135 mm, preferably about 65 mm to about 125 mm, more preferably about 70 mm to about 115 mm and most preferably about 75 mm to about 105 mm; and
(ii) the maximum width W_{b3} of the rear bulb area 300b may range from about 70 mm to about 155 mm, preferably about 80 mm to about 140 mm, more preferably about 85 mm to about 130 mm and most preferably about 90 mm to about 125 mm. In certain embodiments the ratio of W_{b3}/W_{b1} can range from about 1.0 to about 1.8, preferably about 1.0 to about 1.7, and most preferably about 1.0 to about 1.6.

In certain embodiments W_{b1} is about 80 to about 105 mm and W_{b3} is about 85 mm to about 115 mm.

The longitudinal length L_{b} of the backing sheet is about 210 mm to about 320 mm, preferably about 215 mm to about 310 mm, and most preferably about 220 mm to about 300 mm.

In one embodiment:
(i) the longitudinal length from the apex or center of the front edge 302 to the mid-point of the neck region, crotch region, or narrowest region, i.e., minimum width Wₐ₂ of the shaped sanitary napkin, (L_{b front}), is about 70 mm to about 160 mm, preferably about 75 mm to about 155 mm, and most preferably about 80 mm to about 150 mm; and
(ii) the longitudinal length from the apex or center of the rear edge 303 to the mid-point of the neck region, crotch region, or narrowest region, i.e., minimum width Wₐ₂ of the shaped sanitary napkin, (L_{b rear}), is about 90 mm to about 195 mm, preferably about 95 mm to about 190 mm, more preferably about 100 mm to about 185 mm and most preferably about 110 mm to about 180 mm, with the proviso that L_{b rear} is longer than or equal to L_{b front} and preferably L_{b rear} is longer than L_{b front}.

In one embodiment L_{b front} is about 85 mm to about 145 mm and L_{b rear} is about 120 to 160 mm.

As also shown in FIGS. 1 and 2, certain embodiments of the present invention may further comprise two side panels or wings 400 and 400′. The two wings extend laterally from the Top sheet and/or backing sheet in the neck region of the modified hourglass design. In certain embodiments the width W_{b2} of the top sheet and/or backing sheet 300 from the outer most edge of wing 400 to the outer most edge 400′ may range from about 100 mm to about 160 mm, preferably about 110 mm to about 155 mm, and most preferably about 120 mm to about 150 mm. The two side panels or wings 400 and 400′ may also have a longitudinal length of about 30 mm to about 100 mm inches, preferably about 35 mm to about 95 mm, and most preferably about 40 mm to about 90 mm.

In the third aspect of the present invention, the two side panels are present and the width W_{b2} of the top sheet and/or backing sheet 300 from the outer most edge of wing 400 to the outer most edge 400′ may range from about 130 mm to about 160 mm, preferably about 135 mm to about 155 mm, and most preferably about 140 mm to about 150 mm. In addition the width W_{w} from the outer edge of the absorbent core at the point of minimum width Wₐ₂ to the lateral outer most edge of wing 400 or 400′ is about 45 mm to about 55 mm, preferably about 42.5 mm to about 52.5 mm and most preferably about 40 mm to about 50 mm. In the third aspect of the invention, the longitudinal length L_{w} is the length of the wings 400, 400′ that corresponds to the area where the wings attach to or extend from the base of the sanitary napkin and the area in which the wings fold around the crotch of the user's undergarment. L_{w} should range from about 55 mm to about 70 mm, preferably about 50 mm to about 67.5 mm and most preferably about 45 mm to about 65 mm.

The wings 400 and 400′ may be formed in a single or unitary composition from the material comprising the top sheet and/or backing sheet 300. For example the wings 400 and 400′ may be die cut simultaneously with the cutting of the modified hourglass shape for the top sheet and/or backing sheet material 300. The wings 400 and 400′ may be any shape such as oval, rectangular, square, or trapezoidal. The wings 400 and 400′ are sized and shaped to permit the longitudinal folding around the edge of the crotch portion of a user's undergarment, preferably without having to alter the natural path of the edge of the crotch portion of the undergarment.

The backing sheet 300 and/or wings 400 and 400′ may also comprise a means for attaching and/or securing the shaped sanitary napkin of the present invention to the user's undergarment. Typically, the means for attaching comprises an adhesive that is applied to the surface of backing sheet 300 and/or wings 400 and 400′ that is opposite the surface of the sanitary napkin which will be in contact with the user's skin. If an adhesive is employed, a release liner or release paper will cover the adhesive prior to use. The release liner or release paper can be removed, preferably by peeling or pulling, thereby exposing the adhesive prior to application of the sanitary napkin to the user's undergarment. The packaging or wrapping for the individual sanitary napkins may be used in place of a release liner. When the adhesive and the packaging for the individual sanitary napkins are in contact, it is desired to perform a process on the surface of the packaging material in contact with the adhesive to enable exposure of the adhesive and removal of the packaging material without causing a decline in the adhesive power of the adhesive. In this embodiment wherein the packaging material is also used as a release liner for the adhesive, the packaging material may be made from materials such as a siliconized polymeric film. If the side panels or wings are present in this embodiment, the adhesive on the side panels or wings should be protected with a release paper prior to packaging.

The top sheet of the sanitary napkin will be sized and shaped to cover at least the absorbent core 200 described above. The top sheet may also cover all or a portion of the backing sheet 300 and/or wings 400 and 400′. In certain embodiments the top sheet will have substantially the same shape and dimensions as the backing sheet 300 described previously. In certain embodiments the top sheet is secured to the backing sheet 300 and/or absorbent core 200 by any means commonly used in the art for this purpose such as by gluing, crimping, heat sealing, UV-sealing or any combination thereof. The top sheet may also include embossing that may further enhance the aesthetics and/or distribution and removable of body liquids for the outer skin surface of the top sheet into the absorbent core 200. The embossing may be present only on the top sheet or it may extend from the top sheet onto the absorbent core.

### DETAILED DESCRIPTION OF EMBODIMENTS

The following are provided by way of example only and are by no means intended to be limiting

### Example 1

A sanitary napkin comprising a top sheet, a back sheet and an absorbent core in accordance with the present invention was prepared with the following dimensions as indicated in FIG. 2:

Lₐ=length of absorbent core=217 mm

L_{b}=length of back sheet and top sheet=240 mm

L_{w}=length of wing contact=50 mm

Wₐ₁=width of front bulb of absorbent core=76 mm

Wₐ₂=minimum width of absorbent core=45 mm

Wₐ₃=width of rear bulb of absorbent core=76 mm

W_{b1}=width of front back sheet and top sheet front bulb=88 mm

W_{b2}=width of back sheet and top sheet at wings=146 mm

W_{b3}=width of back sheet and top sheet rear bulb=95 mm

W_{w}=width of wing from outer edge of absorbent core to edge of wing=47.5 mm

The foregoing dimensions may vary by ±6%.

FIG. 2 also shows the wing adhesive 410 applied to the garment side of the sanitary napkin and an embossing pattern 500 which has extended from the top sheet onto the absorbent core.

The top sheet was a nonwoven, spunlace sheet comprising 70% hydrophobic cotton and 30% hydrophilic cotton. The top sheet had a basis weight of about 30 gsm.

The back sheet was a bio based polyethylene film, i.e., “green polyethylene”, available from Infiana USA, Inc. of Malvern, Pa., USA.

The absorbent core comprised four layers of nonwoven-carded material comprising 95% hydrophilic organic cotton and 5% acetate copolymer binder. The first and fourth layers of nonwoven-carded material had a width of 76 mm and the second and third layers of nonwoven-carded material sandwiched between the first and fourth layers had a width of about 45 mm. The nonwoven-carded material in each of the four layers had a basis weight of about 72 gsm.

The top sheet was attached to the first layer of the absorbent core nonwoven-carded material using a hot melt adhesive commercially available from Savare Specialty Adhesives, Delaware, Ohio, USA. Similarly the first, second, third and fourth layers of the absorbent core were adhered to each other and the fourth layer of the absorbent core was attached to the backing sheet using the same hot melt adhesive.

The shape of the sanitary napkin described in this example may be further modified without departing from the present invention. For example, the radius of curvature of the front and rear bulb areas may be increased or decreased, the shape of the wings may be varied and the embossing pattern may be adjusted or altered.

### Example 2

A sanitary napkin comprising a top sheet, a back sheet and an absorbent core in accordance with the present invention was prepared using the same materials as described in Example 1 but with the following dimensions:

Lₐ=length of absorbent core=245 mm

L_{b}=length of back sheet and top sheet=272 mm

L_{w}=length of wing contact=60 mm

Wₐ₁=width of front bulb of absorbent core=76 mm

Wₐ₂=minimum width of absorbent core=45 mm

Wₐ₃=width of rear bulb of absorbent core=76 mm

W_{b1}=width of front back sheet and top sheet front bulb=90 mm

W_{b2}=width of back sheet and top sheet at wings=145 mm

W_{b3}=width of back sheet and top sheet rear bulb=103 mm

W_{w}=width of wing from outer edge of absorbent core to edge of wing=47.5 mm

The foregoing dimensions may vary by ±6%.

The shape of the sanitary napkin described in this example may also be further modified without departing from the present invention. For example, the radius of curvature of the front and rear bulb areas may be increased or decreased, the shape of the wings may be varied and the embossing pattern may be adjusted or altered.

The invention illustratively described herein suitably may be practiced in the absence of any element or elements, limitation or limitations which is not specifically disclosed herein. Thus, for example, in each instance herein, any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. An absorbent article comprising a top sheet, a backing sheet and an absorbent core between the top sheet and backing sheet wherein the top sheet comprises about 20 wt % to about 95 wt % of hydrophobic cotton based on the total weight of the top sheet and about 5 wt % to about 80 wt % of hydrophilic cotton based on the total weight of the top sheet.

2. The absorbent article as defined in claim 1 wherein the top sheet comprises: (i) about 30 wt % to about 90 wt % of hydrophobic cotton based on the total weight of the top sheet and about 10 wt % to about 70 wt % of hydrophilic cotton based on the total weight of the top sheet; (ii) about 40 wt % to about 85 wt % of hydrophobic cotton based on the total weight of the top sheet and about 15 wt % to about 60 wt % of hydrophilic cotton based on the total weight of the top sheet or (iii) about 50 wt % to about 80 wt % of hydrophobic cotton based on the total weight of the top sheet and about 20 wt % to about 50 wt % of hydrophilic cotton based on the total weight of the top sheet.

3. The absorbent article as defined in claim 1 wherein the absorbent core comprises at least 50 wt % based on the total weight of the absorbent core of one or more natural or non-synthetic absorbent materials.

4. The absorbent article as defined in claim 3 wherein the one or more natural or non-synthetic absorbent materials comprises cotton.

5. The absorbent article as defined in claim 1 wherein the top sheet has a basis weight of about 20 to about 80 grams per square meter.

6. The absorbent article as defined in claim 1 wherein the center of absorbent core has a basis weight of about 100 to about 500 grams per square meter.

7. The absorbent article of claim 1 wherein the absorbent article is a sanitary napkin, incontinence pad or breast pad.

8. The absorbent article of claim 1 wherein the absorbent article is a sanitary napkin.

9. The sanitary napkin of claim 8 wherein: (i) the top sheet comprises about 50 wt % to about 85 wt % of hydrophobic cotton based on the total weight of the top sheet and about 15 wt % to about 50 wt % of hydrophilic cotton based on the total weight of the top sheet and the top sheet has a basis weight of about 22.5 to about 70 grams per square meter; (ii) the absorbent core comprises at least 90 wt % cotton, is free of any superabsorbent polymeric material and has a basis weight of about 150 to about 450 grams per square meter.

10. A sanitary napkin comprising a top sheet, an absorbent core and backing sheet wherein the absorbent core exhibits a modified hourglass shape comprising a front bulb area with a front terminal end, a rear bulb area with a rear terminal end and a neck area connecting the front bulb area longitudinally opposite the front terminal end and the rear bulb area longitudinally opposite the rear terminal end wherein a maximum width of the front bulb area (Wₐ₁) is about 50 mm to about 110 mm; a maximum width of the rear bulb area (Wₐ₃) is about 50 mm to about 120 mm and a minimum width (Wₐ₂) which is present in the neck area is about 30 mm to about 75 mm with the proviso that Wₐ₃ is greater than or equal to Wₐ₁ and Wₐ₁ and Wₐ₃ are greater than Wₐ₂ and a longitudinal distance from the front terminal end of the front bulb area to Wₐ₂ or a mid-point of the neck area is less than the longitudinal distance from the rear terminal end of the rear bulb area to Wₐ₂ or the mid-point of the neck area.

11. The sanitary napkin of claim 10 wherein: (i) Wₐ₁ is about 55 mm to about 100 mm, about 57 mm to about 95 mm or about 60 mm to about 90 mm; Wₐ₃ is about 55 mm to about 110 mm, about 57 mm to about 105 mm or about 60 mm to about 100 mm; and Wₐ₂ is about 35 mm to about 70 mm, or about 40 mm to about 65 mm.

12. The sanitary napkin of claim 10 wherein the neck area is an elongated neck area that corresponds to a crotch area of a user's undergarment and wherein Wₐ₂ is same or substantially the same in the neck area.

13. The sanitary napkin of claim 10 further comprising at least one wing extending laterally from the top sheet and/or backing sheet on each lateral side of the neck area of the modified hourglass design.

14. The sanitary napkin of claim 13 wherein each wing has a width (W_{w}) wherein W_{w} is a length from an lateral outer edge of the absorbent core at the point of minimum width Wₐ₂ in the neck area to a lateral outer edge of the wing of about 45 mm to about 55 mm.

15. The sanitary napkin of claim 14 wherein W_{w} is about 42.5 mm to about 52.5 mm or about 40 mm to about 50 mm.

16. The sanitary napkin of claim 13 wherein the wings have a longitudinal length (L_{w}) wherein L_{w} is a longitudinal length of the wings where the wings extend from the neck area of the sanitary napkin and is about 55 mm to about 70 mm.

17. The sanitary napkin of claim 16 wherein L_{w} is about 50 mm to about 67.5 mm or about 45 mm to about 65 mm.

18. An sanitary napkin comprising:
(a) a top sheet comprises: (i) about 50 wt % to about 85 wt % of hydrophobic cotton based on the total weight of the top sheet; (ii) about 15 wt % to about 50 wt % of hydrophilic cotton based on the total weight of the top sheet and a basis weight of about 22.5 to about 70 grams per square meter;
(b) a backing sheet,
(c) an absorbent core between the top sheet and backing sheet comprising at least 90 wt % cotton, a basis weight of about 150 to about 450 grams per square and wherein the absorbent core is free of any superabsorbent polymeric material and the absorbent core exhibits a modified hourglass shape comprising a front bulb area with a front terminal end, a rear bulb area with a rear terminal end and a neck area connecting the front bulb area opposite the front terminal end and the rear bulb area opposite the rear terminal end wherein a maximum width of the front bulb area (Wₐ₁) is about 50 mm to about 110 mm; a maximum width of the rear bulb area (Wₐ₃) is about 50 mm to about 120 mm and a minimum width (Wₐ₂) which is present in the neck area is about 30 mm to about 75 mm with the proviso that Wₐ₃ is greater than or equal to Wₐ₁ and Wₐ₁ and Wₐ₃ are greater than Wₐ₂ and a longitudinal distance from the front terminal end of the front bulb area to a mid-point of the neck area that is less than a longitudinal distance from the rear terminal end of the rear bulb area to the mid-point of the neck area; and
(d) at least one wing extending laterally from the top sheet and/or backing sheet of each lateral side of the neck area of the modified hourglass design wherein each wing has a width (W_{w}) wherein W_{w} is a length from a lateral outer edge of the absorbent core at the point of minimum width Wₐ₂ in the neck area to a lateral outer most edge of the wing of about 45 mm to about 55 mm and a longitudinal length (L_{w}) wherein L_{w} is a longitudinal length of each wing where the wing extends from the neck area of the sanitary napkin and is about 55 mm to about 70 mm.
